(19) United States
(12) Patent Application Publication
(10) Pub. No.: US 2005/0181030 A1
(43) Pub. Date: Aug. 18, 2005

(54) Topical stabilized prostaglandin E compound dosage forms

(76) Inventors: Y. Joseph Mo, Princeton, NJ (US); Daniel W. Frank, Broomall, PA (US)

Correspondence Address: OLSON & HIERL, LTD., 20 NORTH WACKER DRIVE, 36TH FLOOR, CHICAGO, IL, 60606, US

(21) Appl. No.: 11/030,506

(22) Filed: Jan. 6, 2005

Related U.S. Application Data
(63) Continuation-in-part of application No. 10/336,481, filed on Jan. 3, 2003, now Pat. No. 6,841,574.

(51) Int. Cl. A61K 31/557; A61L 15/16

(52) U.S. Cl. 424/448; 514/573

(57) ABSTRACT

A packaged, multi-component dosage form comprises a sealed actives compartment containing a prostaglandin E group compound; and a sealed inerts compartment containing a pharmaceutically compatible topical delivery vehicle therefor. Tthe delivery vehicle is combinable with the prostaglandin E group compound to provide a pharmaceutical composition for topical application to a patient, for example, to treat sexual dysfunction.

### CROSS-REFERENCE TO RELATED APPLICATIONS

This application is a continuation-in-part of U.S. patent application Ser. No. 10/336,481, filed on Jan. 3, 2003, now U.S. Pat. No. 6,841,574, which is incorporated herein by reference.

### TECHNICAL FIELD

This application relates to room temperature stable, non-aqueous prostaglandin E compound dosage forms suitable for the treatment of sexual dysfunction in male as well as female patients.

### BACKGROUND OF THE INVENTION

Prostaglandins may exhibit vasodilation or vasoconstriction, smooth muscle stimulation or depression. Prostaglandins of the E group, such as Prostaglandin E₁ (PGE₁) has been reported as having utility for the treatment of sexual erectile dysfunction when injected intracavernously as an aqueous solution in physiological saline, Mahmond et al., J. Urology 147:623-626 (1992), or applied topically. However, the prostaglandins, such as PGE₁, are relatively insoluble in water, and are also relatively unstable. As a result, prostaglandin solutions for injection are prepared shortly prior to use, a relatively inconvenient expedient.

Attempts to stabilize PGE, in aqueous systems by the use of α-cyclodextrin or β-cyclodextrin complexes have been reported. Wiese et al., J. Pharm. Sciences 80:153-156 (1991); Szejtli, J., “Industrial Applications of Cyclodextrins,” Inclusion Compounds III, Academic Press, London, England (1984), pp. 355-368. However, even the aqueous PGE₁ preparations so-stabilized have a relatively short shelf life that limits their practical utilization.

It has now been found that the stability of prostaglandins of the E group can be substantially enhanced without sacrificing bioavailability by the use of specific non-aqueous pharmacologically acceptable compositions that can be stored in a separate compartment from a topical delivery vehicle and combined with the delivery vehicle just prior to use.

### SUMMARY OF THE INVENTION

Prostaglandin E group compounds are stabilized as non-aqueous compositions that include the compound together with a bulking agent that can be a non-aqueous liquid, or a solid in sheet, film, or powder form. Optionally, a skin penetration enhancer can be present.

One embodiment of a packaged, multi-component dosage form of the invention comprises a sealed actives compartment containing a prostaglandin E group compound and a sealed inerts compartment containing a pharmaceutically compatible topical delivery vehicle for the Prostaglandin E group compound, such as prostaglandin E₁, prostaglandin E₂, and/or prostaglandin E₃. The delivery vehicle is combinable with the prostaglandin E group compound to provide a pharmaceutical composition for topical application to a patient. Preferably, the prostaglandin E group compound is substantially uniformly dispersed in a carrier sheet (i.e. a film) within the sealed actives compartment. In one embodiment the carrier sheet is water-soluble. In another embodiment, the carrier sheet is soluble in a physiologically compatible non-aqueous solvent. The topical delivery vehicle is preferably a cream, a gel, or an ointment.

Preferably, at least one of the actives compartment and the inerts compartment contains a skin permeation enhancer, such as an alcohol, a carboxylic acid, a carboxylic ester, a polyol, an amide, a surfactant, a terpene, an alkanone, a solvent, or a combination thereof. Suitable carboxylic ester skin permeation enhancers include, without limitation an N,N-di(C₁-C₈)alkylamino substituted, (C₄-C₁₈)alkyl (C₂-C₁₈)carboxylic ester, a pharmaceutically acceptable addition salt thereof, and a mixture thereof. A preferred N,N-di(C₁-C₈)alkylamino substituted, (C₄-C₁₈)alkyl (C₂-C₁₈)carboxylic ester is dodecyl 2-(N,N-dimethylamino)-propionate or a pharmaceutically acceptable addition salt thereof.

In some embodiments, the prostaglandin E group compound is dispersed in a liquid bulking agent within the actives compartment. Preferably, the liquid bulking agent is an anhydrous alcohol, such as a C₂ to C₄ aliphatic alcohol, benzyl alcohol, or a mixture thereof.

In another embodiment, at least one of the actives compartment and the inerts compartment also contains a viscosity enhancing agent (i.e., a thickening agent).

In one preferred embodiment, the packaged prostaglandin E dosage form comprises a sealed actives compartment containing about 0.025 to 10 parts by weight of a prostaglandin E group compound and a sealed inerts compartment containing about 0.05 to 2.5 parts by weight of a viscosity enhancing agent, about 0.001 to 5 parts by weight of an antifoam agent, about 5 to 75 parts by weight of an alcohol, and about 5 to 75 parts by weight water. Optionally, at least one of the actives compartment and the inerts compartment also contains about 0.5 to 50 parts by weight of a bulking agent. The bulking agent can be a liquid or a solid material. In addition, it is preferred that least one of the actives compartment and the inerts compartment contains about 0.025 to 10 parts by weight of a N,N-di(C₁-C₈)alkylamino substituted, (C₄-C₁₈)alkyl (C₂-C₁₈)carboxylic ester skin permeation enhancer, such as dodecyl 2-(N,N-dimethylamino)-propionate or a salt thereof.

In a preferred embodiment of the dosage forms of the present invention, the actives compartment contains a water-soluble film comprising a prostaglandin E group compound substantially uniformly dispersed in a water-soluble bulking agent. A predetermined size portion of this film can be introduced directly into a moist body cavity to release the prostaglandin compound. Alternatively a predetermined size portion of the sheet or film which includes a prostaglandin compound can be dissolved in an aqueous or non-aqueous solvent that serves as a physiologically compatible delivery vehicle for the prostaglandin compound. For topical applications, the topical delivery vehicle is viscous and substantially non-flowing, such as a cream, gel, or ointment.

In an alternative preferred embodiment a packaged, paired compartment dosage form comprises a sealed actives compartment and a sealed inerts compartment. Compound of prostaglandin E group is contained within the actives compartment, preferably together with a bulking agent, and optionally a skin penetration enhancer. A physiologically compatible viscous topical delivery vehicle is contained within the inerts compartment and is combined with the contents of the actives compartment prior to use, preferably just prior to use. A skin penetration enhancer can be included in the inerts compartment in addition to, or in lieu of, a skin penetration enhancer in the actives compartment.

The present, dosage forms containing a stabilized compound of the prostaglandin E group are useful for amelioration of sexual dysfunction in human patients, e.g., male impotence, premature ejaculation, female sexual arousal disorder, and the like.

### DESCRIPTION OF PREFERRED EMBODIMENTS

Prostaglandin E is a known compound that can be represented by the formula 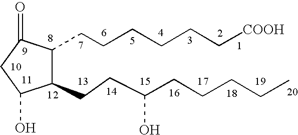
Compounds derived from the foregoing structure and having the 9-oxo, 11α-hydroxy substituents as well as unsaturation in the side chains are known as compounds of the prostaglandin E group, hereinafter collectively referred to as PGE compounds. The compounds of this group include prostaglandin E₁ (PGE₁) represented by the formula 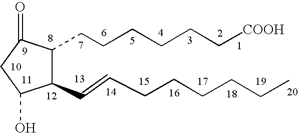
prostaglandin E₂ (or PGE₂) represented by the formula 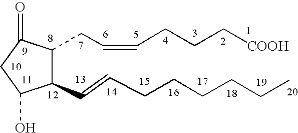
prostaglandin E₃ (or PGE₃) represented by the formula 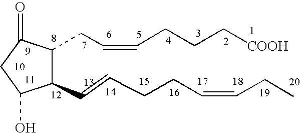
as well as the pharmaceutically acceptable salts thereof.

PGE compounds have useful therapeutic activity as vasodilators and have been utilized to treat male and female sexual disorders, to control lipid metabolism, to treat ulcers, to treat inflammatory skin lesions, and the like therapeutic applications.

PGE compounds are relatively unstable, however, and tend to decompose, especially in aqueous solutions or in an aqueous environment. It has now been found however, that these compounds can be effectively stabilized in non-aqueous media. In some forms of the present invention, sheet-form compositions are provided that can be readily handled and metered to provide convenient dosage forms for topical administration either directly or combined with a viscous topical delivery vehicle such as a cream, gel, ointment, and the like.

PGE compounds can be incorporated as substantially uniformly distributed solids in a sheet-form material, i.e., sheet or film, of a physiologically compatible polymeric material, e.g., a cellulosic ether such as hydroxypropyl cellulose, hydroxypropyl methyl cellulose, and the like, a polysaccharide such as starch, polyvinylpyrrolidone, and the like. Sheet-form materials having a thickness of no more than about 10 mils are commonly referred to as films, and those having a thickness of more than about 10 mils are commonly referred to as sheets. The term “sheet-form” as used herein and in the appended claims refers to sheets as well as films. The sheet-form material can be a solid or a porous material, e.g., a sponge or the like. The sheet-form material containing a PGE compound dispersed therein can be converted into discs, tablets, pellets, and the like, if desired.

These sheet-form articles of manufacture can be water soluble for direct introduction into moist body cavities or soluble in a non-aqueous physiologically compatible solvent for the preparation of a cream or ointment suitable for topical application. The water soluble moiety of the prostaglandin-bearing sheet-form material can also be utilized, of course, for the preparation of aqueous gels based on a polycarbophil, a polyoxyethylene-polyoxypropylene block copolymer, e.g., the so-called poloxamers, and on mixtures thereof, as well as non-aqueous gels based on the polysorbates, liquid block copolymers of propylene oxide and ethylene oxide, and the like.

If desired, the PGE compound-bearing films of the present invention can also include physiologically compatible plasticizers, solubility enhancers (e.g., hydroxypropyl-beta-cyclodextrin), and the like.

These PGE-bearing sheet-form materials can be prepared by first forming a solution of the desired PGE compound in a non-aqueous solvent such as a C₂ to C₄ aliphatic alcohol, e.g., methanol, ethanol, propanol, isopropanol, n-butanol and the like, together with the polymeric material, with or without a skin penetration enhancer, then casting the solution continuously on a roll or batchwise in a shallow dish or pan, and thereafter evaporating the solvent therefrom. The resulting sheet or film has the PGE compound substantially uniformly distributed throughout in an non-aqueous medium that can be readily subdivided and apportioned into desired unit doses each having a predetermined PGE content. The cast sheet or film can also be retained on a solid surface for storage and dissolved immediately prior to use.

The foregoing unit doses preferably are utilized to provide packaged, paired compartment dosage forms in which an actives compartment contains the PGE compound unit dose and an inerts compartment contains the delivery vehicle for a topical application. In the packaged, paired-compartment dosage forms embodying the present invention, the actives compartment can also contain the PGE compound together with a bulking agent in a non-aqueous liquid, particulate or granular form. Suitable liquid bulking agents are silicone oils such as the polydimethylsiloxanes, e.g., cyclomethicone USP, dimethicone USP, and the like, as well as alcohols such as C₂ to C₄ aliphatic alcohols, benzyl alcohol, and the like, or mixtures thereof. Suitable solid bulking agents for this particular purpose are the cyclodextrins such as hydroxypropyl-beta-cyclodextrin, beta cyclodextrin, gamma cyclodextrin, and the like, the polysacharides such as starches, gums, and the like polyvinylpyrrolidone, polyvinyl alcohol, the methyl cellulose derivatives (e.g., hydroxymethyl cellulose), sugars (e.g., lactose), and the like.

A particularly preferred solid dosage form comprises at least one PGE compound, preferably PGE₁, and an amine-substituted carboxylic ester-type skin penetration enhancer, both substantially uniformly distributed in the carrier sheet or admixed with one another in an actives compartment of a packaged paired-compartment dosage form. PGE₁ and PGE₂ are particularly preferred vasoactive agents for the present purposes.

PGE₁ and PGE₂ are well known to those skilled in the art. Reference may be had to various literature references for its pharmacological activities, side effects and normal dosage ranges. See for example, Physician's Desk Reference, 51^{st} Ed. (1997), The Merck Index, 12^{th} Ed., Merck & Co., N.J. (1996), and Martindale The Extra Pharmacopoeia, 28^{th} Ed., London, The Pharmaceutical Press (1982). Prostaglandin E₁ as well as other PGE compounds referenced herein are intended to compass also the pharmaceutically acceptable derivatives thereof, including physiologically compatible salts and ester derivatives.

The quantity of PGE compound, such as PGE₁, present in the solid dosage form is a therapeutically effective amount and necessarily varies according to the desired dose for a particular treatment regimen. The present solid dosage forms can contain about 0.05 to about 25 weight percent of PGE compound, based on the total weight of the composition, preferably about 0.1 to about 15 weight percent of the PGE compound.

A desirable component of the solid dosage form is the skin penetration enhancer. In general, suitable penetration enhancers can be chosen from alcohols, carboxylic acids, carboxylic esters (e.g., amino-substituted carboxylic esters), polyols, amides, surfactants, terpenes, alkanones, solvents (e.g., polar aprotic solvents), and mixtures thereof. See generally Chattaraj, et al., “Penetration Enhancer Classification”, pp. 5-20 in Maibach, et al. (eds.), Percutaneous Penetration Enhancers, CRC Press, Inc., Boca Raton, Fla. (1995), Büiyktimkin, N., et al., “Chemical Means of Transdermal Drug Permeation Enhancement”, in Ghosh, T. K., et al., (eds.) Transdermal and Topical Drug Delivery Systems, Interpharm Press, Inc., Buffalo Grove, Ill. (1997), the relevant disclosures of which are incorporated herein by reference.

Non-limiting examples of suitable alcohols include methanol, ethanol, propanol, butanol, pentanol, hexanol, octanol, nonanol, decanol, 2-butanol, 2-pentanol, benzyl alcohol, caprylic alcohol, decyl alcohol, lauryl alcohol, 2-lauryl alcohol, myristyl alcohol, cetyl alcohol, stearyl alcohol, oleyl alcohol, linolyl alcohol, linolenyl alcohol and mixtures thereof.

Non-limiting examples of suitable carboxylic acids include fatty acids, such as caproic, capric, caprylic, lauric, myristic, palmitic, stearic, isostearic acid, oleic, linoleic, linolenic, and the like; and other straight-chain or branched organic acids, such as valeric, heptanoic, pelargonic, isovaleric, neopentanoic, neoheptanoic, neononanoic, trimethyl hexanoic, neodecanoic and mixtures thereof.

Non-limiting examples of suitable carboxylic esters include sorbitan derivatives, such as sorbitan laurate (SPAN® 20, CRILL™ 1 NF), sorbitan oleate (SPAN® 80, CRILL™ 4 NF), and the like; esters of C₆-C₂₂ carboxylic acid, such as isopropyl myristate, isopropyl palmitate, octyldodecyl myristate, ethyl oleate, ethyl laurate, isopropyl n-hexanoate, isopropyl n-decanoate, isopropyl n-butyrate, methylvalerate, methylpropionate, diethyl sebacate, and the like; and acetates, such as ethyl acetate, butyl acetate, methyl acetate, and the like, and mixtures thereof. Particularly preferred are sorbitan laurate and sorbitan oleate.

Non-limiting examples of suitable polyols include propylene glycol, polyethylene glycol (PEG), ethylene glycol, diethylene glycol, triethylene glycol (TEG), dipropylene glycol, glycerol, propanediol, sorbitol, isosorbitol, dextrans, butanediol, pentanediol, hexanetriol, and mixtures thereof.

Non-limiting examples of suitable surfactants include anionic surfactants, cationic surfactants, nonionic surfactants, amphoteric surfactants, bile salts and lecithin. Suitable anionic surfactants include sodium laurate, sodium lauryl sulfate, and mixtures thereof. Suitable cationic surfactants include cetyltrimethylammonium bromide, tetradecyltrimethylammonium bromide, benzalkonium chloride, octadecyltrimethylammonium chloride, cetylpyridinium chloride, dodecyltrimethylammonium chloride, hexadecyltrimethylammonium chloride, and mixtures thereof. Suitable nonionic surfactants include α-hydro-ω-hydroxypoly(oxyethylene)poly(oxypropyl)poly(oxyethylene) block copolymers, polyoxyethylene ethers, polyoxyethylene sorbitan esters, polyethylene glycol esters of fatty alcohols, and mixtures thereof. Suitable α-hydro-ω-hydroxy-poly(oxyethylene)poly(oxypropyl)poly(oxyethylene) block copolymers include Poloxamers 182, 184, 231, and mixtures thereof. Suitable polyoxyethylene ethers include PEG-4 lauryl ether (BRIJ® 30), PEG-2 oleyl ether (BRIJ® 93), PEG-10 oleyl ether (BRIJ® 96), PEG-20 oleyl ether (BRIJ® 99), and mixtures thereof. Suitable polyoxyethylene sorbitan esters include the monolaurate (TWEEN® 20) the monopalmitate (TWEEN® 40), the monostearate (TWEEN® 60), the monooleate (TWEEN® 80), and mixtures thereof. Suitable polyethylene glycol esters of fatty acids include polyoxyethylene (8) monostearate (MYRJ® 45), polyoxyethylene (30) monostearate (MYRJ® 51), the polyoxyethylene (40) monostearate (MYRJ® 52), and mixtures thereof.

Suitable amphoteric surfactants include, without limitation thereto, lauramidopropyl betaine, cocamidopropyl betaine, lauryl betaine, cocobetaine, cocamidopropylhydroxysultaine, aminopropyl laurylglutamide, sodium cocoamphoacetate, sodium lauroamphoacetate, disodium lauroamphodiacetate, disodium cocoamphodiacetate, sodium cocoamphopropionate, disodium lauroamphodipropionate, disodium cocoamphodipropionate, sodium lauriminodipropionate, disodium cocoamphocarboxymethylhydroxypropylsulfate, and the like.

Particularly preferred carboxylic ester penetration enhancers are amino-substituted carboxylic esters, such as N,N-di(C₁-C₈)alkylamino substituted, (C₄-C₁₈)alkyl (C₂-C₁₈)carboxylic esters or pharmaceutically acceptable acid addition salts thereof. As used herein, the term “(C₄-C₁₈)alkyl (C₂-C₁₈)carboxylic ester” means an ester of a (C₄-C₁₈)alcohol and a (C₂-C₁₈)carboxylic acid. The term “N,N-di(C₁-C₈)alkylamino substituted,” in reference to a (C₄-C₁₈)alkyl (C₂-C₁₈)carboxylic ester means that either the alcohol portion or the carboxylic acid portion from which the ester is prepared bears an amino substituent NRₓR_{y}, wherein Rₓ and R_{y} are each independently a (C₁-C₈)alkyl group. Preferably Rₓ and R_{y} are both methyl groups.

Preferred N,N-di(C₁-C₈)alkylamino substituted, (C₄-C₁₈)alkyl (C₂-C₁₈)carboxylic esters are dodecyl-2-(N,N-dimethylamino)-propionate (DDAIP); dodecyl-2-(N,N-dimethylamino)-acetate (DDAA); 1-(N,N-dimethylamino)-2-propyl dodecanoate (DAIPD); 1-(N,N-dimethylamino)-2-propyl myristate (DAIPM); 1-(N,N-dimethylamino)-2-propyl oleate (DAIPO); and pharmaceutically acceptable acid addition salts thereof. Particularly preferred is DDAIP, alone or in combination with an auxiliary permeation enhancer. DDAIP is available from Steroids, Ltd. (Chicago, Ill.). The preparation of DDAIP and crystalline acid addition salts thereof is described in U.S. Pat. No. 6,118,020 to Büyüktimkin, et al., which is incorporated herein by reference. Long chain similar amino substituted, alkyl carboxylic esters can be synthesized from readily available compounds as described in U.S. Pat. No. 4,980,378 to Wong, et al., which is incorporated herein by reference to the extent that it is not inconsistent herewith. Such amino-substituted carboxylic ester penetration enhancers are also sometimes referred to as alkyl-2-(N-substituted amino)-alkanoates and (N-substituted amino)-alkanol alkanoates. For convenient reference, alkyl-2-(N-substituted amino)-alkanoates and (N-substituted amino)-alkanol alkanoates can be grouped together under the term alkyl (N-substituted amino) esters.

Non-limiting examples of solvents include aliphatic esters, such as triethylcitrate (TEC), and triacetin; aromatic esters, such as diethylphthalate (DEP); dipolar aprotic solvents, such as N-methyl-2-pyrrolidone (NMP), diethylene glycol monoethyl ether (DGME, transcutol), isosorbide dimethylether (DMI), dimethyldecylphosphoxide, methyloctylsulfoxide, dimethyllaurylamide, dodecylpyrrolidone, dimethylacetamide, dimethylsulfoxide, decylmethylsulfoxide, and dimethylformamide; oils, such as squalane, and octanol, and the like which affect keratin permeability.

Particularly preferred skin permeation enhancers are dipolar aprotic solvents, particularly NMP, DGME, and DMI; aliphatic esters, particularly TEC, and triacetin; carboxylic esters that are sorbitan derivatives, particularly sorbitan laurate (SPAN® 20), and sorbitan oleate, N,N-di(C₁-C₈)alkylamino substituted C₄-C₁₈)alkyl (C₂-C₁₈)carboxylic esters, particularly DDAIP, and combinations thereof.

The penetration enhancer is present in an amount sufficient to enhance the penetration of the PGE compound into tissue. The specific amount varies necessarily according to the desired release rate and specific form of PGE compound used. Generally, this amount is in the range of about 0.01 percent to about 20 percent, based on the total weight of the composition to be administered to a patient.

The desired release rate, including controlled or sustained release of the active compound can also be modulated by selection of the topical delivery vehicle, e.g., a hydrophobic vehicle such as polydimethylsiloxanes and the like. Carboxy-terminated polydimethylsiloxanes can also enhance skin permeation by the active compound.

Natural and modified polysaccharide gums can also be present, for example as a viscosity enhancing agent, as part of the carrier sheet or the topical delivery vehicle. Suitable representative gums are the natural and modified galactomannan gums. A galactomannan gum is a carbohydrate polymer containing D-galactose and D-mannose units, or other derivatives of such a polymer. There is a relatively large number of galactomannans, which vary in composition depending on their origin. The galactomannan gum is characterized by a linear structure of β-D-mannopyranosyl units linked (1→4). Single membered α-D-mannopyranosyl units, linked (1→6) with the main chain, are present as side branches. Galactomannan gums include guar gum, which is the pulverized endosperm of the seed of either of two leguminous plants (Cyamposis tetragonalobus and psoraloids) and locust bean gum, which is found in the endosperm of the seeds of the carob tree (Ceratonia siliqua). Suitable modified polysaccharide gums include ethers of natural or substituted polysaccharide gums, such as carboxylmethyl ethers, ethylene glycol ethers and propylene glycol ethers.

Other suitable representative gums include agar gum, carrageenan gum, ghatti gum, karaya gum, rhamsan gum and xanthan gum. The composition of the present invention may contain a mixture of various gums, or mixture of gums and acidic polymers.

Gums, and galactomannan gums in particular, are well-known materials. See for instance, Industrial Gums:Polysaccharides & Their Derivatives, Whistler R. L. and BeMiller J. N. (eds.), 3^{rd} Ed. Academic Press (1992) and Davidson R. L., Handbook of Water-Soluble Gums and Resin, McGraw-Hill, Inc., N.Y. (1980). Most gums are commercially available in various forms, commonly a powder, and ready for use in food and topical compositions. For example, locust bean gum in powdered form is available from Tic Gums Inc. (Belcam, Md.).

When present, the polysaccharide gums are present in the range of about 0.1 percent to about 5 percent, based on the total weight of the composition, with the preferred range being in the range of about 0.5 percent to 3 percent. In one preferred embodiment, about 2.5 percent by weight of a polysaccharide gum is present.

An optional alternative to the polysaccharide gum is a polyacrylic acid polymer. A common variety of polyacrylic acid polymer is known generically as “carbomer.” Carbomer is polyacrylic acid polymers lightly cross-linked with polyalkenyl polyether. It is commercially available from the B. F. Goodrich Company (Akron, Ohio) under the designation “CARBOPOL™.” A particularly preferred variety of carbomer is that designated as “CARBOPOL 940.”

Other polyacrylic acid polymers suitable for use are those commercially available under the designation “PEMULEN™” (B.F. Goodrich Company) and “POLYCARBOPHIL™” (A. H. Robbins, Richmond, Va.). The PEMULEN™ polymers are copolymers of C₁₀ to C₃₀ alkyl acrylates and one or more monomers of acrylic acid, methacrylic acid or one of their simple esters crosslinked with an allyl ether of sucrose or an allyl ether of pentaerythritol. The POLYCARBOPHIL™ product is polyacrylic acid cross-linked with divinyl glycol.

The concentration of lipophilic compound required necessarily varies according to other factors such as the desired semi-solid consistency and the desired skin penetration promoting effects. Suitably the concentration of lipophilic compound is the range of about 0.5 percent to about 40 percent by weight based on the total weight of the composition. The preferred topical composition contains lipophilic compound in the range of about 7 percent to about 40 percent by weight based on the total weight of the composition.

Where a mixture of aliphatic alcohol and alphatic ester are employed, the suitable amount of alcohol is in the range of about 0.5 percent to about 75 percent. In one preferred embodiment, the amount of alcohol is in the range of about 5 percent to about 15 percent, while that of aliphatic ester is in the range of about 2 percent to about 15 percent (again based on the total weight of the composition). In another preferred embodiment, the amount of alcohol is in the range of about 0.5 percent to about 10 percent, while that of aliphatic ester is in the range from zero percent to about 10 percent (again based on the total weight of the composition).

An optional, but preferred, component is an emulsifier. A suitable emulsifier generally will exhibit a hydrophilic-lipophilic balance number greater than 10. Sucrose esters, and specifically sucrose stearate, can serve as emulsifiers for the composition. Sucrose stearate is a well-known emulsifier available from various commercial sources. When an emulsifier is used, sucrose stearate, present in an amount up to about 2 percent, based on the total weight of the composition, is preferred. The preferred amount of sucrose stearate emulsifier can also be expressed as a weight ratio of emulsifier to polysacharide gum.

Other suitable emulsifiers are the polyoxyethylene sorbitan esters, long chain alcohols, preferably cetostearyl alcohol, and fatty acid glycerides. Suitable polyoxyethylene sorbitan esters include the monolaurate (TWEEN 20, SPAN 20) the monopalmitate (TWEEN 40), the monostearate (TWEEN 60), and the monooleate (TWEEN 80) and mixtures thereof. Preferred fatty acid glycerides include glyceryl monooleate, triolean, trimyristin and tristearin.

Another optional ingredient is an antifoam agent, a chemical that reduces the tendency of the finished preparation to generate foam on shaking or agitation. Silicones are the preferred antifoam agents; however, a wide variety of alcohols and lipids exhibit similar properties. With the exception of alcohols, the selected antifoam agent must be effective in relatively small concentrations, and are employed in trace amounts. Illustrative antifoam agents are dimethicone, cetyl dimethicone, dimethicone silylate, dimethiconol, a mixture of dimethicone and hydrated silica, isopropyl alcohol, hexyl alcohol, trimethylsiloxysilicate, triphenyl trimethicone and the like. Particularly preferred antifoam agent is a mixture of dimethicone with an average chain length of 200 to 300 dimethylsiloxane units and hydrated silica, commercially available under the designation SIMETHICONE USP from Dow Corning Corporation, Michigan.

The composition can include a buffer system, if desired. Buffer systems are chosen to maintain or buffer the pH of compositions within a desired range. The term “buffer system” or “buffer” as used herein refers to a solute agent or agents which, when in a water solution, stabilize such solution against a major change in pH (or hydrogen ion concentration or activity) when acids or bases are added thereto. Solute agent or agents which are thus responsible for a resistance or change in pH from a starting buffered pH value in the range indicated above are well known. While there are countless suitable buffers, potassium phosphate buffers (e.g., potassium phosphate monohydrate, KH₂PO₄ N.F., and the like) have proven effective for compositions of the present invention and are preferred.

The final pH value of the pharmaceutical composition may vary within the physiological compatible range. Necessarily, the final pH value is one not irritating to human skin and preferably such that transdermal transport of the PGE compound is facilitated. Without violating this constraint, the pH may be selected to improve PGE compound stability and to adjust consistency when required. In one embodiment, the preferred pH value is about 3.0 to about 7.4, more preferably about 3.0 to about 6.5, most preferably from about 3.5 to about 6.0.

For preferred topical delivery vehicles the remaining component of the composition is an aqueous composition, such as a solution or gel. Preferably, the water present in the composition is purified, e.g., deionized water. Such delivery vehicle compositions contain water in the range of more than about 50 to about 95 percent, based on the total weight of the composition. The specific amount of water present is not critical, however, being adjustable to obtain the desired viscosity (usually about 50 cps to about 10,000 cps) and/or concentration of the other components. The topical delivery vehicle preferably has a viscosity of at least about 30 centipoise. Viscosity enhancing agents can be included to afford the desired level of viscosity.

PGE compound stabilizers and excipients, such as organic acids and alcohols, cyclodextrins, coloring agents, rheological agents, and preservatives can be added to the extent that they do not limit penetration of the PGE compound.

The ingredients listed above may be combined in any order and manner that produces a stable composition for ultimately receiving the PGE compound, such as PGE₁ and the like, preferably substantially evenly dispersed throughout. One available approach to preparing such compositions involves evenly dispersing the polysaccharide gum (or polyacrylic acid) in a premixed water/buffer solution and then thoroughly homogenizing (i.e., mixing) the resulting mixture. When present, the emulsifier is added to the water/buffer solution before dispersing the polysaccharide gum. Any suitable method of adjusting pH value to the desired level may be used, for example, by adding concentrated phosphoric acid or sodium hydroxide.

The PGE compound, with or without a penetration enhancer, is then combined therewith prior to use with mixing. The resulting composition is ready for topical, intrameatal, or vaginal administration.

These compositions can be used for prolonged treatment of peripheral vascular disease, male impotency and other disorders treated or treatable by PGE compounds while avoiding low bioavailability and rapid chemical decomposition associated with other delivery methods.

One preferred embodiment of the invention is a solid, dissolvable prostaglandin E dosage form which comprises a prostaglandin E group compound substantially uniformly dispersed in a water-soluble film. The film is produced by casting a film from an admixture comprising (a) about 0.025 to 10 parts by weight prostaglandin E₁; (b) about 0.55 to 50 parts by weight hydroxypropyl-β-cyclodextrin; (c) about 0.025 to 10 parts by weight dodecyl 2-(N,N-dimethylamino)-propionate or a salt thereof; (d) about 0.05 to 25 parts by weight hydroxypropyl methylcellulose; (e) about 0.05 to 25 parts by weight polyethylene glycol 8000; (f) about 0.001 to 5 parts by weight silicone antifoam agent; (g) about 5 to 90 parts by weigh water; and (h) about 5 to 75 parts by weight ethanol.

In another embodiment, a preparation ready for administration comprises about 0.01 percent to about 5 percent modified polysaccharide gum; about 0.001 percent to about 1 percent of a PGE compound, preferably PGE₁, or a pharmaceutically acceptable salt thereof, a lower alkyl ester thereof and mixtures thereof; about 0.5 percent to about 10 percent dodecyl 2-(N,N-dimethylamino)-propionate or a salt thereof; about 0.5 percent to about 10 percent of a lower alcohol selected from the group consisting of ethanol, propanol, isopropanol and mixtures thereof; about 0.5 percent to about 10 percent on an ester selected from the group consisting of ethyl laurate, isopropyl myristate, isopropyl laurate and mixture thereof; based on the weight of the preparation, together with an acid buffer. Preferably the preparation also comprises up to about 2 percent by weight sucrose stearate.

Variations in the treating compositions which do not adversely affect the effectiveness of the PGE compound will be evident to one skilled in the art, and are within the scope of this invention. For example, additional ingredients such as coloring agents, anti-microbial preservatives, emulsifiers, lubricants, perfumes, PGE compound stabilizers, and the like, may be included as long as the resulting preparation retains desirable properties, as described above. When present, preservatives are usually added in amounts of about 0.05 to about 0.30%. Suitable preservatives include methylparabens (methyl PABA), propylparabens (propyl PABA) and butylhydroxy toluene (BHT). Suitable perfumes and fragrances are known in the art; a suitable fragrance is up to about 5 percent and fragrances are known in the art; a suitable fragrance is up to about 5 percent myrtenol, preferably about 2 percent myrtenol, based on the total weight of the composition. The compositions of the present invention can also include a small amount, about 0.01 to about 4 percent by weight, of a topical anesthetic, if desired. Typical topical anesthetics include lidocaine, benzocaine, dyclonine, dibucaine, pharmaceutically acceptable salts and mixtures thereof. In one preferred embodiment, the topical anesthetic is about 0.5 percent dyclonine, based on the weight of the composition.

Illustrative two-compartment dosage forms are set forth below:

| | Amount, parts by weight | |
|---|---|---|
| | Preferred | More Preferred |
| Actives Compartment | | |
| PGE₁ | 0.025-10 | 0.05-0.5 |
| Dodecyl 2-(N,N-dimethylamino)- | 0.025-10 | 0.05-2.5 |
| propionate.HCl | | |
| Lactose | 1-50 | 2.5-10 |
| Inerts Compartment | | |
| Hydroxypropyl methyl cellulose | 0.05-2.5 | 1-6 |
| Silicone antifoam agent | 0.001-5 | 0.1-2 |
| Hydroxypropyl-β-cyclodextrin | 0.5-25 | 1-10 |
| Water (deionized or U.S.P.) | 5-75 | 20-60 |
| Ethanol | 5-75 | 20-60 |

If desired, preservatives such as methyl paraben, propyl paraben, benzalkonium chloride, benzethonium chloride, and the like, can be included as well.

Yet another two-compartment dosage form is set forth below:

| | Amount, parts by weight |
|---|---|
| Actives Compartment | |
| PGE₁ | 0.2 |
| Dodecyl 2-(N,N-dimethylamino)- | 2.5 |
| propionate.HCl | |
| Ethanol, anhydrous, USP | 5 |
| Inerts Compartment | |
| Guar gum | 2.5 |
| Ethyl laurate | 3 |
| Water, USP, buffered to pH 5.5* | 100 |
| with 0.1M KH₂PO₄ (N.F.) and NaOH | |

Illustrative two-part compositions for casting a PGE₁-containing film are set forth below.

| | Amount, parts by weight | |
|---|---|---|
| | Preferred | More Preferred |
| Part A | | |
| PGE₁ | 0.025-10 | 0.05-0.5 |
| Dodecyl 2-(N,N-dimethylamino)- | 0.025-10 | 0.05-2.5 |
| propionate.HCl | | |
| Hydroxypropyl-β-cyclodextrin | 0.05-25 | 1-10 |
| Part B | | |
| Hydroxypropyl methylcellulose | 0.05-25 | 1-6 |
| Polyethylene glycol 8000 powder | 0.05-25 | 0.5-5 |
| Silicone antifoam agent | 0.001-5 | 0.1-2 |
| Hydroxypropyl-β-cyclodextrin | 0.5-25 | 1-10 |
| Water (deionized or U.S.P.) | 5-90 | 20-60 |
| Ethanol | 5-75 | 20-60 |

Parts A and B are combined with agitation, the resulting mixture is cast as a layer on a surface, and the ethanol is permitted to evaporate to produce a sheet-form material, i.e., either a sheet or a film depending upon the thickness of the cast layer.

The present invention is further illustrated by the following examples.

### EXAMPLE 1

### TWO COMPARTMENT PACKAGED DOSAGE FORM

A viscous topical delivery vehicle was prepared by combining hydroxypropyl methyl cellulose (2 grams; METHOCEL® E4M; Dow Chemical Co.), polyethylene glycol 8000 powder (0.5 grams), deionized water (97.5 grams), and a trace amount of an antifoam agent (SIMETHICONE®; Dow Corning Corp., Midland, Mich.).

First an aliquot of deionized water (about 25 grams) was heated to about 80° C., and then the hydroxypropyl methyl cellulose (2 grams) was added thereto with stirring until dissolved. A trace amount of the anti-foam agent was added to the resulting hot solution.

Polyethylene glycol powder (0.5 grams; PEG 8000, was added to cold deionized water (50 grams) with stirring until dissolved to produce a cold polyethylene glycol solution.

The obtained cold and hot solutions were combined with stirring, more deionized water was added to the combined solution (q.s. 100 grams), and the produced solution was placed in an ice bath and chilled to below about 30° C. with continuous agitation. The pH value of the produced solution was measured as 6.25.

This solution is suitable as constituent for the inerts compartment of the two-compartment dosage form. Ethyl alcohol can be added to produce a solution suitable for casting a sheet-form unit dose such as a film or sheet.

The contents for the actives compartment was prepared by admixing dry prostaglandin E₁ (0.018 grams) and dodecyl 2-(N,N-dimethylamino)-propionate (0.12 grams).

The actives content prepared as described hereinabove was then combined with three grams of the inerts composition described above to which anhydrous ethyl alcohol (3 grams) was added.

A clear, viscous gel was obtained, suitable for topical or intrameatal administration. The pH value of the obtained gel was measured as 4.5.

### EXAMPLE 2

### FILM WITH PGE₁ AND SKIN PERMEATION ENHANCER

A portion of the clear gel produced as described in Example 1 was spread on a glass panel with a 6-mil film spreader and dried for several hours until a film was produced. Upon the addition of a small amount of water (100 milligrams) a one-inch square of film reconstituted into a clear gel within about 15 seconds.

### EXAMPLE 3

### FILM WITH PGE₁

PGE₁ powder (0.024 grams) was combined with an aqueous solution having the following constituents:

| | |
|---|---|
| Hydroxypropyl methyl cellulose | 0.06 grams |
| PEG 8000 powder | 0.015 grams |
| Deionized water | 2.925 grams |
| Ethyl alcohol, anhydrous | 3 grams |

and prepared in the same manner as described in Example 1, above. The resulting combination of PGE₁ and the aqueous solution was shaken vigorously for 15 to 30 seconds until the PGE₁ went into solution.

The resulting solution was poured onto a glass panel and dried at ambient temperature for about 3.5 hours. A film containing PGE₁ substantially uniformly dispersed therein was obtained.

### EXAMPLE 4

### FILM WITH PGE₁ AND DODECYL 2-(N,N-DIMETHYLAMINO)-PROPIONATE

The procedure of Example 3, above, was used to dissolve PGE₁ (0.024 grams) and dodecyl 2-(N,N-dimethylamino)-propionate (0.03 grams) in an aqueous solution having the following constituents:

| | |
|---|---|
| Hydroxypropyl methyl cellulose | 0.06 grams |
| PEG 8000 powder | 0.015 grams |
| Deionized water | 2.9 grams |
| Ethyl alcohol, anhydrous | 3 grams |

The obtained solution was poured onto a glass panel, spread with a 6-mil. film spreader, and dried for about 3.5 hours. A dry film containing substantially uniformly dispersed PGE₁ and dodecyl 2-(N,N-dimethylamino)-propionate was obtained. The film was readily water miscible. If desired, the film can be packaged in a sealed actives compartment along with a sealed inerts compartment containing a pharmaceutically compatible topical delivery vehicle for the material. Suitable delivery vehicles are materials that are combinable with the prostaglandin E group compound for topical application to a patient, as described hereinabove. The film can be cut and packaged into individual doses for application with individually packaged quantities of delivery vehicle. Multiple, individually packaged doses of the film and of the delivery vehicle can be packaged together, if desired, in the form of a multi-dose kit.

The foregoing examples have been provided as an illustration of preferred embodiments of the invention, and are not meant to limit the scope of the invention.

1. A packaged, multi-component dosage form comprising a sealed actives compartment containing a prostaglandin E group compound; and a sealed inerts compartment containing a pharmaceutically compatible topical delivery vehicle therefor; the delivery vehicle being combinable with the prostaglandin E group compound to provide a pharmaceutical composition for topical application to a patient.

2. The dosage form of claim 1 wherein the prostaglandin E group compound is substantially uniformly dispersed in a carrier sheet within the sealed actives compartment.

3. The dosage form of claim 2 wherein the carrier sheet is water-soluble.

4. The dosage form of claim 2 wherein the carrier sheet is soluble in a physiologically compatible non-aqueous solvent.

5. The dosage form of claim 1 wherein the prostaglandin E group compound is selected from the group consisting of prostaglandin E₁, prostaglandin E₂, and prostaglandin E₃.

6. The dosage form of claim 1 wherein the topical delivery vehicle is selected from the group consisting of a cream, a gel, and an ointment.

7. The dosage form of claim 1 wherein at least one of the actives compartment and the inerts compartment also contains a skin permeation enhancer.

8. The dosage form of claim 7 wherein the skin permeation enhancer is selected from the group consisting of an alcohol, a carboxylic acid, a carboxylic ester, a polyol, an amide, a surfactant, a terpene, an alkanone, a solvent, and a combination thereof.

9. The dosage form of claim 8 wherein the carboxylic ester skin permeation enhancer is selected from the group consisting of an N,N-di(C₁-C₈)alkylamino substituted, (C₄-C₁₈)alkyl (C₂-C₁₈)carboxylic ester, a pharmaceutically acceptable addition salt thereof, and a mixture thereof.

10. The dosage form of claim 9 wherein the skin permeation enhancer comprises dodecyl 2-(N,N-dimethylamino)-propionate or a pharmaceutically acceptable addition salt thereof.

11. The dosage form of claim 1 wherein the prostaglandin E group compound is dispersed in a liquid bulking agent within the actives compartment.

12. The dosage form of claim 11 wherein the liquid bulking agent is an anhydrous alcohol.

13. The dosage form of claim 12 wherein alcohol is selected from the group consisting of a C₂ to C₄ aliphatic alcohol, benzyl alcohol, and a mixture thereof.

14. The dosage form of claim 1 wherein at least one of the actives compartment and the inerts compartment also contains a viscosity enhancing agent.

15. The dosage form of claim 1 wherein the sealed actives compartment contains about 0.025 to 10 parts by weight of a prostaglandin E group compound; and the sealed inerts compartment contains about 0.05 to 2.5 parts by weight of a viscosity enhancing agent, about 0.001 to 5 parts by weight of an antifoam agent, about 5 to 75 parts by weight of an alcohol, and about 5 to 75 parts by weight water.

16. The dosage form of claim 15 wherein the actives compartment also contains about 0.5 to 50 parts by weight of a solid bulking agent.

17. The dosage form of claim 15 wherein the actives compartment also contains about 0.5 to 50 parts by weight of a liquid bulking agent.

18. The dosage form of claim 15 wherein at least one of the actives compartment and the inerts compartment contains about 0.025 to 10 parts by weight of an N,N-di(C₁-C₈)alkylamino substituted, (C₄-C₁₈)alkyl (C₂-C₁₈)carboxylic ester or a pharmaceutically acceptable addition salt thereof.

19. A prostaglandin E dosage form comprising:
(a) about 0.01 percent to about 5 percent modified polysaccharide gum;
(b) about 0.001 percent to about 1 percent of a prostaglandin E compound or a pharmaceutically acceptable salt thereof, a lower alkyl ester thereof, or a mixture thereof;
(c) about 0.5 percent to about 10 percent dodecyl 2-(N,N-dimethylamino)-propionate or a salt thereof;
(d) about 0.5 percent to about 10 percent of an alcohol selected from the group consisting of ethanol, propanol, isopropanol and a mixture thereof;
(e) about 0.5 percent to about 10 percent on an ester selected from the group consisting of ethyl laurate, isopropyl myristate, isopropyl laurate, and a mixture thereof;
(f) an acid buffer; and
(g) up to about 2 percent by weight sucrose stearate.

20. A film cast from the dosage form of claim 19.

21. A solid, dissolvable prostaglandin E dosage form which comprises a compound of prostaglandin E group substantially uniformly dispersed in a water-soluble film, wherein the film is produced by casting a film from an admixture comprising:
(a) about 0.025 to 10 parts by weight prostaglandin E₁;
(b) about 0.55 to 50 parts by weight hydroxypropyl-β-cyclodextrin;
(c) about 0.025 to 10 parts by weight dodecyl 2-(N,N-dimethylamino)-propionate or a salt thereof;
(d) about 0.05 to 25 parts by weight hydroxypropyl methylcellulose;
(e) about 0.05 to 25 parts by weight polyethylene glycol 8000;
(f) about 0.001 to 5 parts by weight silicone antifoam agent;
(g) about 5 to 90 parts by weigh water; and
(h) about 5 to 75 parts by weight ethanol.

22. A water-soluble film comprising a prostaglandin E group compound substantially uniformly dispersed in a film containing a water-soluble bulking agent.
